SEQUENCE LISTING (69 sequences)

SEQ ID NO: 1 - protein, 10 aa, synthetic construct
APNHLLEVRV

SEQ ID NO: 2 - protein, 12 aa, synthetic construct
LTAYVVKVFSLA

SEQ ID NO: 3 - protein, 14 aa, synthetic construct
DDLKQLANGVDRYI

SEQ ID NO: 4 - protein, 15 aa, synthetic construct
SDKKGICVADPFEVT

SEQ ID NO: 5 - protein, 10 aa, synthetic construct
SDAGLTFTSS

SEQ ID NO: 6 - protein, 16 aa, synthetic construct
SDKKGICVADPCFEVT

SEQ ID NO: 7 - protein, 16 aa, synthetic construct
VQAERSGIPIVTSPYQ

SEQ ID NO: 8 - protein, 13 aa, synthetic construct
QALPYSTVGNSNL

SEQ ID NO: 9 - protein, 12 aa, synthetic construct
NIFLKDSITTWE

SEQ ID NO: 10 - protein, 12 aa, synthetic construct
LHLSVRTLELRP

SEQ ID NO: 11 - protein, 9 aa, synthetic construct
QRSYTVAIA

SEQ ID NO: 12 - protein, 9 aa, synthetic construct
KRPQDAKNT

SEQ ID NO: 13 - protein, 9 aa, synthetic construct
ERLGREGVQ

SEQ ID NO: 14 - protein, 14 aa, synthetic construct
LNCQRYYGGGGYST

SEQ ID NO: 15 - protein, 10 aa, synthetic construct
LSRKVLLDGV

SEQ ID NO: 16 - protein, 12 aa, synthetic construct
ESASLRSEETKV

SEQ ID NO: 17 - protein, 12 aa, synthetic construct
GQWKIRAYYENS

SEQ ID NO: 18 - protein, 12 aa, synthetic construct
IAVHYLDETEQW

SEQ ID NO: 19 - protein, 11 aa, synthetic construct
LRLPYVVREQL

SEQ ID NO: 20 - protein, 13 aa, synthetic construct
TAYVVKVFSLAVN

SEQ ID NO: 21 - protein, 423 aa, synthetic construct
MRAPAQIFGFLLLLFPGTCFAHHGGEHFEGEKVFRVNVEDENHINIIRELASTTQIDFWKPDSVTQIKPHSTVDFRVKAEDTVTVENVLKQNELQYKVLISNLRNVVEAQFDSRVRANGHSYEKYNKWETIEAWTQQVATENPALISRSVIGTTFEGRAIYLLKVGKAGQNKPAIFMDCGFHAREWISPAFCQWFVREAVRTYGREIQVTELLDKLDFYVLPVLNIDGYIYTWTKSRFWRKTRSTHTGSSCIGTDPNRNFDAGWCEIGASRNPCDETYCGPAAESEKETKALADFIRNKLSSIKAYLTIHSYSQMMIYPYSYAYKLGENNAELNALAKATVKELASLHGTKYTYGPGATTIYPAAGGSDDWAYDQGIRYSFTFELRDTGRYGFLLPESQIRATCEETFLAIKYVASYVLEHLY

SEQ ID NO: 22 - protein, 422 aa, synthetic construct
MRAPAQIFGFLLLLFPGTCFAFQSGQVLAALPRTSRQVQVLQNLTTTYEIVLWQPVTADLIVKKKQVHFFVNASDVDNVKAHLNVSGIPCSVLLADVEDLIQQQISNDTVSGRASASYYEQYHSLNEIYSWIEFITERHPDMLTKIHIGSSFEKYPLYVLKVSGKEQTAKNAIWIDCGIHAREWISPAFCLWFIGHITQFYGIIGQYTNLLRLVDFYVMPVVNVDGYDYSWKKNRMWRKNRSFYANNHCIGTDLNRNFASKHWCEEGASSSSCSETYCGLYPESEPEVKAVASFLRRNINQIKAYISMHSYSQHIVFPYSYTRAKSKDHEELSLVASEAVRAIEKTSKNTRYTHGHGSETLYLAPGGGDDWIYDLGIKYSFTIELRDTGTYGFLLPERYIKPTCREAFAAVSKIAWHVIRNV

SEQ ID NO: 23 - protein, 18 aa, synthetic construct
LRTWSRRATRSTKTLKVV

SEQ ID NO: 24 - protein, 21 aa, synthetic construct
  source 1..21: note = This sequence is part of a branched peptide.
  SITE 19..20: note = Sites of linkage in octameric dendromer
LRTWSRRATRSTKTLKVVKKV

SEQ ID NO: 25 - protein, 22 aa, synthetic construct
  source 1..22: note = This sequence is part of a branched peptide.
  SITE 19..21: note = Sites of linkage in octameric dendromer
LRTWSRRATRSTKTLKVVKKKV

SEQ ID NO: 26 - protein, 18 aa, synthetic construct
DYGHYDDKDTLDLNTPVD

SEQ ID NO: 27 - protein, 16 aa, synthetic construct
HWPFGGAACSILPSLI

SEQ ID NO: 28 - protein, 14 aa, synthetic construct
CALSVDRYRAVASW

SEQ ID NO: 29 - protein, 15 aa, synthetic construct
CALSVDRYRAVASWC

SEQ ID NO: 30 - protein, 16 aa, synthetic construct
  source 1..16: note = This sequence is part of a branched peptide.
  SITE 13..15: note = Sites of linkage in octameric dendromer
QGIGIPLITAEIKKKV

SEQ ID NO: 31 - protein, 21 aa, synthetic construct
LNLCALSVDRYRAVASWSRVI

SEQ ID NO: 32 - protein, 21 aa, synthetic construct
IVRSWSAVARYDSVLACLNLV

SEQ ID NO: 33 - protein, 24 aa, synthetic construct
CALSVDRYRAVASWGIPLITAIEI

SEQ ID NO: 34 - protein, 22 aa, synthetic construct
VLNLCALSVDRYGAVASWSRVI

SEQ ID NO: 35 - protein, 22 aa, synthetic construct
VLNLCAGSVDRYRAVASWSRVI

SEQ ID NO: 36 - protein, 22 aa, synthetic construct
VLNLGALSVDRYRAVASWSRVI

SEQ ID NO: 37 - protein, 5 aa, synthetic construct
  VARIANT 1: note = D-tryptamine
  REGION 1..5: note = Cyclic peptide
  SITE 2: note = D-aspartic acid
  SITE 4: note = D-valine
XDPVL

SEQ ID NO: 38 - protein, 508 aa, synthetic construct
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQASRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVR

SEQ ID NO: 39 - protein, 514 aa, synthetic construct
MRAPAQIFGFLLLLFPGTCFACCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 40 - protein, 309 aa, synthetic construct
MRAPAQIFGFLLLLFPGTCFAQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCHRWPESPKAQASSVPTAQPQAEGSLAKATAPATTRNTGRGGEEKKKEKEKEEQEERETKTPECPQCYNCPNPTADCKTAVNCSSDFDACLITKAELGYHYVAQAGRRQSSHFSLLKCWDYRCEPSHWPHCPYFNWGYKCITSVGSLSIAISTTSQPA

SEQ ID NO: 41 - protein, 361 aa, synthetic construct
CCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRR

SEQ ID NO: 42 - protein, 132 aa, synthetic construct
QQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 43 - protein, 19 aa, synthetic construct
  source 1..19: note = This sequence is part of a branched peptide.
  SITE 19: note = Site of linkage in dendromer
LRTWSRRATRSTKTLKVVK

SEQ ID NO: 44 - protein, 18 aa, synthetic construct
  source 1..18: note = This sequence is part of a branched peptide.
LRTWSRRATRSTKTLKVV

SEQ ID NO: 45 - protein, 20 aa, synthetic construct
  source 1..20: note = This sequence is part of a branched peptide.
  SITE 19..20: note = Sites of linkage in dendromer
LRTWSRRATRSTKTLKVVKK

SEQ ID NO: 46 - protein, 14 aa, synthetic construct
  source 1..14: note = This sequence is part of a branched peptide.
  SITE 13..14: note = Sites of linkage in dendromer
QGIGIPLITAEIKK

SEQ ID NO: 47 - protein, 13 aa, synthetic construct
  source 1..13: note = This sequence is part of a branched peptide.
  SITE 13: note = Site of linkage in dendromer
QGIGIPLITAEIK

SEQ ID NO: 48 - protein, 12 aa, synthetic construct
  source 1..12: note = This sequence is part of a branched peptide.
QGIGIPLITAEI

SEQ ID NO: 49 - protein, 417 aa, Homo sapiens
MLALLVLVTVALASAHHGGEHFEGEKVFRVNVEDENHINIIRELASTTQIDFWKPDSVTQIKPHSTVDFRVKAEDTVTVENVLKQNELQYKVLISNLRNVVEAQFDSRVRATGHSYEKYNKWETIEAWTQQVATENPALISRSVIGTTFEGRAIYLLKVGKAGQNKPAIFMDCGFHAREWISPAFCQWFVREAVRTYGREIQVTELLDKLDFYVLPVLNIDGYIYTWTKSRFWRKTRSTHTGSSCIGTDPNRNFDAGWCEIGASRNPCDETYCGPAAESEKETKALADFIRNKLSSIKAYLTIHSYSQMMIYPYSYAYKLGENNAELNALAKATVKELASLHGTKYTYGPGATTIYPAAGGSDDWAYDQGIRYSFTFELRDTGRYGFLLPESQIRATCEETFLAIKYVASYVLEHLY

SEQ ID NO: 50 - protein, 15 aa, synthetic construct
MLALLVLVTVALASA

SEQ ID NO: 51 - protein, 423 aa, Homo sapiens
MKLCSLAVLVPIVLFCEQHVFAFQSGQVLAALPRTSRQVQVLQNLTTTYEIVLWQPVTADLIVKKKQVHFFVNASDVDNVKAHLNVSGIPCSVLLADVEDLIQQQISNDTVSPRASASYYEQYHSLNEIYSWIEFITERHPDMLTKIHIGSSFEKYPLYVLKVSGKEQTAKNAIWIDCGIHAREWISPAFCLWFIGHITQFYGIIGQYTNLLRLVDFYVMPVVNVDGYDYSWKKNRMWRKNRSFYANNHCIGTDLNRNFASKHWCEEGASSSSCSETYCGLYPESEPEVKAVASFLRRNINQIKAYISMHSYSQHIVFPYSYTRSKSKDHEELSLVASEAVRAIEKTSKNTRYTHGHGSETLYLAPGGGDDWIYDLGIKYSFTIELRDTGTYGFLLPERYIKPTCREAFAAVSKIAWHVIRNV

SEQ ID NO: 52 - protein, 14 aa, synthetic construct
QKKIEEIAAKYKHS

SEQ ID NO: 53 - protein, 13 aa, synthetic construct
KYKHSVVKKSDGA

SEQ ID NO: 54 - protein, 17 aa, synthetic construct
RAARISLGPRSIKAFTE

SEQ ID NO: 55 - protein, 14 aa, synthetic construct
LRANISHKDMQLGR

SEQ ID NO: 56 - protein, 510 aa, synthetic construct
MAPFEPLASGILLLLWLICGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 57 - protein, 18 aa, synthetic construct
MAPFEPLASGILLLLWLI

SEQ ID NO: 58 - protein, 21 aa, synthetic construct
MPGLGRRAQWLCWWWGLLCSC

SEQ ID NO: 59 - protein, 360 aa, synthetic construct
CGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRR

SEQ ID NO: 60 - protein, 132 aa, synthetic construct; identical to SEQ ID NO: 42

SEQ ID NO: 61 - protein, 4 aa, synthetic construct
RRQQ

SEQ ID NO: 62 - protein, 4 aa, synthetic construct
RRQA

SEQ ID NO: 63 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 64 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 65 - protein, 20 aa, synthetic construct
MPGLGRRAQWLCWWWGLLCS

SEQ ID NO: 66 - protein, 21 aa, synthetic construct
MRAPAQIFGFLLLLFPGTCFA

SEQ ID NO: 67 - protein, 63 aa, Homo sapiens
RWPESPKAQASSVPTAQPQAEGSLAKATAPATTRNTGRGGEEKKKEKEKEEQEERETKTPECP

SEQ ID NO: 68 - protein, 93 aa, Homo sapiens
QCYNCPNPTADCKTAVNCSSDFDACLITKAELGYHYVAQAGRRQSSHFSLLKCWDYRCEPSHWPHCPYFNWGYKCITSVGSLSIAISTTSQPA

SEQ ID NO: 69 - skipped

(19) United States
(12) Patent Application Publication
(10) Pub. No.: US 2024/0335507 A1
(43) Pub. Date: Oct. 10, 2024

(54) THERAPEUTIC COMPOSITION FOR USE IN THE TREATMENT OF COVID-19 AND OTHER CYTOKINE STORM ASSOCIATED DISORDERS

(71) Applicant: Kelsius Laboratories LLC, Baltimore, MD (US)

(72) Inventor: Lajos BARANYI, Gaithersburg, MD (US)

(73) Assignee: Kelsius Laboratories LLC, Baltimore, MD (US)

(21) Appl. No.: 18/590,419

(22) Filed: Feb. 28, 2024

Related U.S. Application Data
(63) Continuation of application No. 16/898,378, filed on Jun. 10, 2020, now Pat. No. 11,918,624.

(51) Int. Cl. A61K 38/17; A61K 38/18; A61K 38/48; A61K 45/06; C12N 15/86

(52) U.S. Cl. CPC A61K 38/1709; A61K 38/1875; A61K 38/4813; C12N 15/86; A61K 45/06; C12Y 304/17002

(57) ABSTRACT

It is disclosed a therapeutic composition for use in the treatment of COVID-19 and other cytokine storm associated disorders, wherein the therapeutic composition comprises at least one active agent being selected from the following active agent groups a) to e):
a) complement factor 3-targeting inhibitor of complement activation cascade
b) carboxypeptidase B enzyme
c) complement factor 5a receptor-targeting inhibitor of complement activation cascade
d) endothelin A receptor-targeting inhibitor of extravasation
e) bone morphogenic protein.

It is further disclosed a method of treating COVID-19 and other cytokine storm associated disorders, wherein said method comprises administering an effective amount of at least one active agent being selected from the above mentioned active agent groups a) to e).

### CROSS-REFERENCE TO PRIOR APPLICATIONS

This application is a continuation of U.S. patent application Ser. No. 16/898,378, filed Jun. 10, 2020, which contents are incorporated herein in their entirety by reference.

The present invention is directed to a therapeutic composition for use in the treatment of COVID-19 and other cytokine storm associated disorders.

### REFERENCE TO A SEQUENCE LISTING

The instant application contains a Sequence Listing XML which has been submitted electronically and is hereby incorporated by reference in its entirety. Said Sequence Listing XML copy, created on May 31, 2024, is named “222552000101USST26” and is 72,013 bytes in size.

### FIELD OF THE INVENTION

Pathogenic corona viruses (SARS, MRSA, SARS-nCov2) are single positive RNA strand viruses with a wide spectrum of hosts and a strong ability to mutate at high rate, spread rapidly by waste, contacts and aerosols, and only a very limited set of treatments are available to protect infected patients.

The “corona-like” virus structure itself consists of the positive strand RNA packaged into the core of the virus, said core comprising membrane proteins and the spike protein. Membrane proteins support the viral morphogenesis, and the spike protein is a glycoprotein that attaches the virion to the cell membrane by interacting with the host receptor, thereby initiating the infection of the cells.

### PRIOR ART

Clearly, the spike proteins as well as the other viral proteins are under intense study and huge effort is made to develop new rational antiviral treatment as well as vaccines. Currently discussed, antiviral treatment and drug candidates are: Remdesivir (viral polymerase inhibitor), Umifenovir (Fusion inhibitor), Lopinavir (Retrovirus protease inhibitor), Ritonavir (Retrovirus protease inhibitor) and Hydroxychloroquine (anti malaria drug).

A common factor in all of the known antiviral drugs is that they are highly toxic. This is tolerable as long as the application is life saving or preventing a severely debilitating outcome of the diseases. In many cases, however, the viral disease symptoms that may trigger the treatment are mild and may remain mild, rendering the toxic antiviral drug too dangerous to apply, or the side effects in severe cases of the viral infection actually further hurt the patients that already suffer from lung, liver, and/or intestinal symptoms.

### OBJECT

It is an object of the present invention to provide another therapeutic approach. Particularly the inventor analysed the known mechanism of the viral infection, the progress of the infection, and the response of the human body. Critical elements in the physiological response were identified in order to design new drug candidates that shall help the body to hinder the progress of the viral infection, stopping the debilitating side effects and consequences of the disease.

### INVENTION

Disclosed herein is a therapeutic composition for use in the treatment of COVID-19 and other cytokine storm associated disorders, wherein the therapeutic composition comprises at least one active agent being selected from the following active agent groups a) to e):
a) complement factor 3-targeting inhibitors,
b) carboxypeptidase B enzymes,
c) complement factor 5a receptor-targeting inhibitors,
d) endothelin A receptor-targeting inhibitors,
e) bone morphogenetic proteins.

Further disclosed is a method of treating COVID-19 and other cytokine storm associated disorders, wherein said method comprises administering an effective amount of at least one active agent being selected from the abovementioned active agent groups a) to e).

As used herein, the term “therapeutic composition” refers to a composition to be administered as a therapeutic treatment to a subject suffering from a disorder.

As used herein, the term “treatment” in the context of the administration to a subject refers to the reduction or inhibition of the progression and/or duration of disorder.

As used herein, the term “subject” refers to a mammal, most preferably a human.

As used herein, the term “composition” refers to an effective amount of one or more active agents, optionally in combination with pharmaceutical acceptable carriers and/or excipients.

By “pharmaceutically acceptable,” is meant a compound that is not biologically or otherwise undesirable, that is, the compound may be incorporated into a topical formulation of the invention and administered to a patient without causing any undesirable biological effects or interacting in a deleterious manner with any of the other components of the formulation in which it is contained.

As used herein, the term “active agent” refers to a compound being effective in the treatment of a disorder, when being administered in an effective amount to a subject suffering from a disorder.

As used herein, the term “effective amount” refers to the amount of a treatment that is sufficient to result in the prevention of the development, recurrence, or onset of a condition being associated with a cytokine storm, and one or more symptoms thereof, to enhance or improve the prophylactic effect(s) of another therapy, reduce the severity, the duration of a cytokine storm associated disorder, ameliorate one or more symptoms of a condition being associated with a cytokine storm, prevent the advancement or recurrence of a cytokine storm, cause regression of a disorder being associated with a cytokine storm.

As used herein, the term “cytokine storm associated disorder” refers to a disorder that may result in a condition called “cytokine storm”. The cytokine storm is characterized by unusual high blood levels of cytokines, such as chemokines, interferons, interleukins, lymphokines as compared to the corresponding blood levels of said cytokines in a healthy subject. Particularly relevant are the blood levels of inflammatory cytokines interleukin-1 (IL-1), IL-6, IL-12, and IL-18, tumor necrosis factor alpha (TNF-α), interferon gamma (IFNγ), granulocyte-macrophage colony stimulating factor (GM-CSF) and Transforming Growth Factor (TGF).

If the blood level of at least one inflammatory cytokine is raised in a subject by more than 10%, more than 20% or even more than 30% as compared to the corresponding blood level(s) of said cytokine(s) in a healthy subject, this is an indicator for an onset or for the presence of a cytokine storm. If the blood level of two or more inflammatory cytokines is raised in a subject by more than 10%, more than 20% or even more than 30% as compared to the corresponding blood level(s) of said cytokine(s) in a healthy subject, this is an even more clear indicator for an onset or for the presence of a cytokine storm.

Cytokine storm may be induced by various instances, including but not limited to infection with SARS COV-2 virus, any other corona viruses and any hemorrhagic fever causing viruses. It may also be induced by other physiological states and disorders.

The term “cytokine storm associated disorder” includes any disorder of a subject, that may result in a “cytokine storm” to occur. Examples for cytokine storm associated disorders that may be treated in accordance with the present invention are COVID-19, Hemorrhagic fever diseases, Allergy, Alzheimer's disease in CNS, ARDS/respiratory distress, Bechet's disease, bronchial asthma, capillary leak syndrome, ischemia, ischemia-reperfusion, chronic lung disease, immunocomplex glomerulonephritis, Churg-Strauss syndrome, Cystic fibrosis, Mediterranean fever Guillain-Barre, Cardiac ischemia and infarct reperfusion, Kimura's disease, Multiple Sclerosis, MS Meningitis Pancreatitis, Preeclampsia, Retinitis, Allogenic graft rejection, chronic inflammation, septic shock, acute ischemia, trauma and surgery, cardiac pathology and multiple organ failure, allotransplantation, asthma, acute respiratory distress syndrome, arthus reaction, burns injuries, Crohn's disease, dermatomyositis, drug-induced pseudoallergy, Forssman shock, glomerulonephritis, hemolytic anemia, hemorrhagic shock, hemodialysis reactions, hereditary angeoderma, Huntington's disease, infertility as a result of spontaneous abortion, ischemia reperfusion injuries, human complex-induced vasculitis, multiple sclerosis, myasthenia gravis, Pick's disease, paroxysmal nocturnal hemoglobinuria, post-bypass syndrome, psoriasis, rheumatoid arthritis and septic shock.

### BRIEF DESCRIPTION OF THE FIGURES

FIG. 1 shows the carboxypeptidase B enxzyme activity in plasma, activated plasma, cytokine storm plasma and activated cytokine storm plasma.

FIG. 2 shows the C5a receptor inhibitor activity of the peptide PR226MAP.

FIG. 3 shows the expression cassette designed for the recombinant human carboxypeptidase preproprotein expression.

FIG. 4 shows the expression cassette designed for the recombinant bone morphogenetic protein.

FIG. 5 shows that an injection of 1 μg/kg PR226MAP peptide does not induce any cardiovascular change but abrogates the lethal effects of the subsequent challenge with a lethal dose of C5a.

FIG. 6 shows that the animal survived 7 injections but developed acute hemorrhage and succumbed by the third hour displaying all the symptoms of acute cytokine storm.

FIG. 7A shows the severity of blood pressure decrease in rats treated with the C5a receptor inhibitor peptide.

FIG. 7B shows the severity of bradycardia in rats treated with the C5a receptor inhibitor peptide.

FIG. 8A shows the effective inhibition of smooth muscle contraction in femoral artery by treatment with the inventive peptides capable of blocking ET-A receptors.

FIG. 8B shows the effective inhibition of smooth muscle contraction in carotid artery by treatment with the inventive peptides capable of blocking ET-A receptors.

### DETAILED DESCRIPTION OF INVENTION

The complement system is a part of the immune system. Over 30 proteins and protein fragments make up the complement system, including serum proteins, and cell membrane receptors. When stimulated by one of several triggers, proteases in the system cleave specific proteins to release cytokines and initiate an amplifying cascade of further cleavages. The end result of this complement activation or complement fixation cascade is stimulation of phagocytes to clear foreign and damaged material, inflammation to attract additional phagocytes, and activation of the cell-killing membrane attack complex.

Any process that activates the complement system goes through a series of positive feedback loops. Complement activation releases anaphylatoxins, C3a, C4a and C5a, all of which are powerful signaling molecules. The C5a gets mobilized, activated and released a substantial number of mediators. These factors in turn cause pulmonary hypertension, vascular dilation, together with the membrane attack complex cause vascular leakage, hypotension, hypoxia, acidosis, vasoconstriction, more hypoxia, intestinal symptoms and bacterial leakage, endotoxemia, fever, more complement activation, more granulocyte activation, more endothelial damage, and finally severe pulmonary hemorrhage, and cardiovascular damage. The major mediators of this process are the cytokines. Therefore, this process is also known as cytokine storm.

Once this cytokine storm is fully developed, it is irreversible, and most of the cases causes multiple organ failure, and death. Cytokine storm plays key role in cardiovascular-pulmonary collapse that is central element in death caused by hemorrhagic viruses, blood lost induced hemorrhagic shock, bacterial septic shock, pancreatitis, and most of systemic inflammatory shock diseases, drug-adverse reactions, CAR-T cell therapy adverse reactions. Further, it is known to be one of the reasons for many fatal cases of COVID-19.

The cytokine storm being a devastating medical disorder, it is disclosed a novel and effective treatment that reduces, diminishes and abolishes the devastating physiological effects of the cytokine storm. The goal of the treatment is avoiding immunosuppression while the cytokine storm is limited and remains at levels compatible with healing from the disease state that induced it.

Particularly, it is disclosed a treatment directed to a set of 5 targets participating in eliciting, amplifying and otherwise exacerbating the cytokine storm. These targets were rationally selected with expertise from hundreds of molecules participating in the onset and amplification of the cytokine storm.

It is disclosed a therapeutic composition for use in the treatment of COVID-19 and other cytokine storm associated disorders. By comprising at least one active agent selected from the following active agent groups:
a) complement factor 3-targeting inhibitors,
b) carboxypeptidase B enzymes,
c) complement factor 5a receptor-targeting inhibitors,
d) endothelin A receptor-targeting inhibitors,
e) bone morphogenetic proteins.

the therapeutic composition of the present invention provides means for effectively treating COVID-19 and other cytokine storm associated disorders. In some embodiments of the invention the therapeutic composition used comprises at least two, three, four or five distinct active agents being selected from at least two, three, four or five of the active agent groups a) to e).

### a) Complement Factor 3-Targeting Inhibitor of Complement Activation Cascade

The root cause of the cytokine storm is the rapid and out of control complement system activation and while the triggering factors counts in dozens, and each of them result in a protease activity called C3 convertase (embodied in various forms) sharing common feature to be capable of specifically cleaving the C3 (complement factor 3) into C3a and C3b components, creating an active C3b, that itself acts as a C3 convertase via the C3b cleavage product. Targeting C3 and preventing the positive feedback loop is the first target selected.

Therefore, the present invention encompasses a therapeutic composition comprising at least one complement factor 3-targeting inhibitor of complement activation cascade. In some embodiments the complement factor 3-targeting inhibitor is a direct inhibitor of the complement factor 3 receptor. In other embodiments the complement factor 3-targeting inhibitor is an inhibitor of the complement factor 3 receptor activity through reducing the release of C3a anaphylatoxin. By reducing the release of C3a anaphylatoxins the activity of the complement factor 3 receptor can be reduced indirectly, that is, without using direct complement receptor 3 inhibitors.

C3 is the central element of the complement activiation pathway, and blocking excess C3 activation blocks all the anaphylatoxin production, including subsequent raise of the cytokine storm. However, according to one aspect of the invention, C3 and its activity shall not be eliminated completely, as it is essential for the protection from microbes and for development of proper immune reaction. This is why in some embodiments the complement factor 3-targeting inhibitor is an inhibitor of the complement factor 3 receptor activity through reducing the release of C3a anaphylatoxin. This allows better fine tuning of complement factor 3 receptor activity.

In order to provide a complement inhibitor that acts fast, reversibly and without accumulation of toxic byproduct, the inventor has analyzed the human C3 protein with a proprietary algorithm implemented in C language and designed a series of peptides that were tested for their ability for inhibiting complement activity in a hemolysis assay. The inventor found that out of a library of 19 peptides 7 peptides inhibited the complement of complement mediated hemolysis very strong (80% or higher inhibition), 4 peptides inhibited hemolysis strong (40% or higher inhibition), 5 peptides had moderate inhibitor activity (20% or higher inhibition) and 4 peptides had low inhibitor activity (less than 20% inhibition) under the test conditions.

Accordingly, in some embodiments of the inventive therapeutic composition the complement factor 3-targeting inhibitor of complement activation cascade of active agent group a) is selected from the following group of peptides:

AP1314, LTA1309, DDL1619, SDK1606, SDA984, SDK1709, VQA1857, QAL1363, NIF1464, LHL1431, QRS1008, KRP1057, ERL1041, LNC1538, LSR1099, ESA1331, GQW1513, IAV1501, LRL1384 and TAY1409

having the amino acid sequences as specified in SEQ IDs Nos. 1-20.

The one letter code amino acid sequences and the hemolysis inhibition properties of said peptides are indicated in Table 1 of the examples further below.

### b) Carboxypeptidase B Enzyme

Whenever there is complement activation, the cleavage of key components C3, C4 and C5 results in massive release of respective anaphylatoxins. Each of them is a single chain, 74 amino acid long amino terminal fragment of the respective complement factor (complement factor 3, 4 and 5), and each of them is terminated with an arginine amino acid at their carboxyterminal end. While all of them are powerful mediators, their activity is strongly diminished by the loss of the carboxy terminal arginine.

C5a is an effective chemoattractant, initiating accumulation of phagocytic cells at sites of infection or recruitment of antigen-presenting cells to lymph nodes. C5a also modulates the balance between activating versus inhibitory IgG Fc receptors on leukocytes by increasing it, thereby enhancing the (auto) immune response and triggering a genuine cytokine storm.

The anaphylatoxins and many of the messengers amplifying cytokine storm are peptides with an amino acid arginine at the carboxy terminus. The enzyme that removes that arginine renders the specific messengers and anaphylatoxins lose or reduce or change their biological activity. However, the inventor has found that this enzyme activity is very low in plasma. What is more, it is rapidly depleted as infection, hemorrhage, hypoxia develops, blood coagulation triggered (see Examples further below), in each case when systemic complement activation occurs, resulting in allowing anaphylatoxins to act at their full potential to trigger the positive feedback that results in Cytokine storm.

For removing the terminal arginine of peptides there is a set of enzymes called carboxypeptidase (E.C. numbers: 3.4.16 through 3.4.18), a subgroup consists of basic carboxypeptidases that specialize in removing basic amino acids Arg and Lys from the carboxy terminus of the peptide. It turned out that there is one enzyme specialized for converting C5a into desArg C5a. What is more, it has been shown that it is rapidly lost by self-inactivation.

Replenishing this enzyme, is expected to protect the patient from the excess power of these mediators, coagulation system malfunctions and granulocyte leukocyte activation and chemoattraction to the shock organs. Therefore, the present invention encompasses a therapeutic composition comprising at least carboxypeptidase B enzyme.

In some embodiments of the inventive therapeutic composition, said composition comprises as an active agent carboxypeptidase B enzyme being selected from the carboxypeptidases EC 3.4.16-3.4.18.

Further, the inventor has developed recombinant carboxypeptidase B enzymes having the amino acid sequences as specified in SEQ IDs No. 21-22. Said enzymes may be used in the treatment of cytokine associated disorders such as COVID-19. Therefore, in some embodiments of the inventive therapeutic composition said composition comprises as an active agent one of the aforementioned recombinant carboxypeptidase B enzymes. The one letter code amino acid sequences of said enzymes are indicated in the examples further below.

Said recombinant carboxypeptidase B enzyme can be obtained by a method comprising the following steps:
firstly introducing plasmids with recombinant carboxypeptidase B protogene sequence into Escherichia coli cells to construct a recombinant engineering strain,
fermenting for inducing and expressing recombinant carboxypeptidase B protogene (proenzyme),
additional renaturation, pancreatin conversion as well as separation, and
purification to obtain carboxypeptidase B enzyme.

The present application also encompasses a nucleic acid encoding recombinant carboxypeptidase B enzyme. Further, it is disclosed a lentiviral vector comprising this nucleic acid. In addition, it is disclosed a plasmid comprising this nucleic acid and a cell line comprising said plasmid. Furthermore, it is disclosed a method of producing recombinant carboxypeptidase B enzyme by cultivating a cell comprising the plasmid.

### c) Complement Factor 5a Receptor-Targeting Inhibitor of Complement Activation Cascade

By using one or both of the aforementioned active agents of groups a) and b), it is possible to control complement activation through inhibiting complement factor 3 and adding Carboxypeptidase B enzyme to keep the desArg form of the newly generated anaphylatoxins and disturbance in the coagulation system. This could significantly reduce the chance of the viral infection to turn into full cytokine storm.

However, a prolonged release and accumulation of C5a, even a very minuscule amounts on the site of the virus infection or tissue damage would still be able to activate attract and granulocytes and cause massive degranulation in the affected tissue, causing irreversible tissue damage. The C5a acting on its receptor is the main culprit in this process. It has been found that this receptor is one of the most sensitive receptors in cell signaling. The inventor has noted that picomolar quantities of C5a can trigger the intracellular activation signal and increase of Ca++ levels.

C5a is the most powerful of the anaphylatoxins, and it exerts massive effect on cell expressing its receptor. These cells are heavily involved in the cytokine storm and induce the production of a vast array of secondary messengers, regardless of C5a has or lacks the terminal arginine. The nature of the effect may change though, but if the receptor is blocked, the overactivation of the cells involved in triggering the cytokine storm may be blocked, and kept at acceptable level, not threatening the patient's life or tissue integrity.

Therefore, the present invention encompasses a therapeutic composition comprising at least one complement factor 5a receptor-targeting inhibitor of complement activation cascade involved in the cytokine storm induction. In some embodiments the complement factor 5a receptor-targeting inhibitor is an inhibitor of the complement factor 5a receptor. In some embodiments the complement factor 5a receptor-targeting inhibitor is a competitor of the complement factor 5a or a fragment thereof. In some embodiments the complement factor 5a targeting inhibitor is not a competitor for the binding sites for the complement factor 5a, but interacts in another way with the complement factor 5a receptor, thereby inhibiting complement activation cascade. The inventor has synthesized and tested a large number of peptide-based C5aR agonists and antagonists, both, in vitro and in vivo. However, in many cases the beneficial effects do not materialize to the required extent. Particularly, some of the inhibitory variants of the peptides are not sufficiently compete with naturally occurring level of C5a in vivo for the ligand binding. In some embodiments of the present invention, the complement factor 5a receptor-targeting inhibitor of complement activation cascade of active agent group c) is selected from the group of peptides:
LRT2463, LRT10353, LRT20707, DYG2094 and HWP1667

having the amino acid sequences as specified in SEQ IDs No. 23-27.

The one letter code amino acid sequences of said peptides are indicated in Table 3 of the examples further below. Said peptides have been shown to be very effective inhibitors of complement factor 5a receptor, both, in vitro and in vivo.

These peptides are considered potentially useful in clinical applications, but the observed biphasic activity of the most promising one (PR226), that it acted as inhibitor at high concentration and activator as low concentration, was a property that questioned the safety of the peptide for human use. However, the inventor has designed an oligomeric form of the peptide, a dendrimer, that increased its activity 10,000-fold, and the receptor inhibitory activity increased from femtomolar to 200 nM levels, a tremendous potential.

The activity in vivo, in rat and pig models, is shown in the examples below. Further, it has been shown that the peptide can be delivered in vivo very safely, even at very high concentration. The reason is that the peptide immediately binds to the erythrocyte surfaces and forms a stable membrane bound pool that is completely inert to erythrocytes, but upon contact with C5aR bearing cells, the inhibitory effect is fully exhibited. In addition, in the membrane bound state, the peptide remains active for at least 12 hours. Therefore, it is a very effective C5aR inhibitor.

### d) Endothelin A Receptor-Targeting Inhibitor of Extravasation

The inventor has observed that activated macrophages and leukocytes do not do any harm while in the circulation. They do not act until they are surrounded by live, healthy erythrocytes. Particularly, they need to undergo extravasation, moving out of the capillaries into the affected tissue parenchyma.

Further, the inventor has identified that endothelin receptor is one of the bottleneck elements in the very complex and highly redundant process of extravasation. Particularly, the inventor noted that the process of extravasation is controlled by the endothelial cells. They open the gates upon the signal delivered by the endothelin peptide and engaging the endothelin A receptor on their surface. If said gates are not opened, the activated leukocytes cannot pass through the tightly bound endothelial membrane.

Therefore, the inventor has picked this as the fourth target in the treatment of cytokine storm. Particularly, the inventor has postulated that blocking extravasation could be the solution to down regulate the overactivated cells and prevent them from causing irreversible tissue damage.

Therefore, the present invention encompasses a therapeutic composition comprising at least one endothelin A receptor-targeting inhibitor of cytokine storm activation cascade. In some embodiments the endothelin A receptor-targeting inhibitor is an inhibitor of the endothelin A receptor. In some embodiments the endothelin A receptor-targeting inhibitor is a competitor of the endothelin A or a fragment thereof. In some embodiments the endothelin A targeting inhibitor interacts in another way with the endothelin A receptor and/or the endothelin A or a fragment thereof, thereby inhibiting complement activation cascade.

In some embodiments the endothelin A receptor-targeting inhibitor of extravasation is selected from the group of peptides:
CAL1595, CAL1698, QGI10797, LNL2390, IVR2333, CAL2614, VLN2390, VLN2433, VLN2443 and TDP610

having the amino acid sequences as specified in SEQ IDs No. 28-37.

The one letter code amino acid sequences of said peptides are indicated in Table 4 of the examples further below.

### e) Bone Morphogenetic Protein (BMP)

Complete blocking of the cytokine storm may not be advised, especially in case of viral and bacterial infection, as it could result in an iatrogenic immunodeficiency, and allow the infectious agents to propagate uncontrolled, causing a devastating outcome of the disease.

The inventor has opted for two elements to remedy this issue: the first is that they have designed and selected effector molecules that do not accumulate and break down rapidly, allowing a rapid tuning of the status of the cytokine storm; and the second element is that an additional target should be included that helps to heal the internal wound, a tissue damage accompanied by cell death, tissue disorganization to be restored in an enhanced and supported manner, as well as delay the invasion on fibroblast, preventing the irreversible tissue scarring that lead to loss of function in the affected organ.

Viral cytotoxicity, the system of tissue specific general complement activation, the passing of granulocytes through the endothelial layers from the capillaries into the shock organ parenchyma and cytokine-induced endothelial disfunction individually and combined, cause massive loss of shock organ parenchyma cells, blood vessel integrity, and hemorrhage. In simple terms, this is a mechanism of making wounds inside the affected tissues in cytokine storm. With loss of tissue integrity, the organ becomes dysfunctional and in extreme cases that causes death. In less severe cases, the patients undergo a long process of wound healing. That process is slow, requires rebalancing of cytokines, and orchestrates the activity of stem cells, cell differentiation as well as invasion by various cell types, including fibroblasts. Unlike “standard” wounds, hemorrhaged tissues heal very slowly.

Fibroblasts appear very early in the hemorrhagic wounds, and cause fibrosis, scar formation that implies significant function loss in any affected organ. Fibrosis denotes excessive scarring, which exceeds the normal wound healing response to injury in tissues injured during cytokine storm.

It has been shown that bone morphogenetic proteins may regulate excess fibrosis. Therefore, in some embodiments of the present invention the therapeutic composition comprises a bone morphogenetic protein. In specific embodiments said bone morphogenetic protein is selected from bone morphogenetic protein BMP-6.

Further, there are disclosed new recombinant bone morphogenetic proteins BMP and new active domains of bone morphogenetic proteins, and the specifications of said new recombinant bone morphogenetic proteins and the specifications of said new active domains of bone morphogenetic proteins are indicated in Tables 5 and 6 of the examples further below.

Therefore, in some embodiments of the present invention the therapeutic composition comprises a new recombinant bone morphogenetic protein BMP having an amino acid sequence as specified in one of the SEQ ID Nos. 38 to 40.

In further specific embodiments said recombinant bone morphogenetic protein is selected from a protein comprising an active domain having an amino acid sequence as specified in one of the SEQ ID Nos. 41 or 42 or from a protein comprising both of the two active domains having the amino acid sequences as specified in SEQ ID Nos. 41 and 42. Said protein may comprise in addition the specific leader sequences, hinge regions and anchor fragments as specified in table 6 below.

The present application also encompasses a nucleic acid encoding the recombinant bone morphogenetic proteins BMP. Further, it is disclosed a lentiviral vector comprising this nucleic acid. In addition, it is disclosed a plasmid comprising this nucleic acid and a cell line comprising said plasmid. Furthermore, it is disclosed a method of producing recombinant bone morphogenetic protein BMP by cultivating a cell comprising the plasmid.

In addition to the claimed composition, the present invention also encompasses a rationally designed method of treating a cytokine storm associated disorder. Particularly, it is disclosed a method of treating a cytokine storm associated disorder, characterized in that the method comprises administering an effective amount of at least one active agent selected from active agent groups a) to e):
a) complement factor 3-targeting inhibitors,
b) carboxypeptidase B enzymes,
c) complement factor 5a receptor-targeting inhibitors,
d) endothelin A receptor-targeting inhibitors,
e) bone morphogenetic proteins.

The inventive method of treatment is a selection of a limited number or target molecules within the highly complex cascades of interrelated enzymes, mediators, and receptor and other proteins that when operating in concert, lead to the phenomenon known as cytokine storm.

Unlike general immune suppressions, depletion of complements system, plasmapheresis, irradiation and other interventions already in practice, this treatment leaves intact the functioning of the immune system, innate and antigen specific, essential to protection from the underlying viral or bacterial or fungal infections while enabling the fine tuning of the level of the functions necessary for the protective immunity by designing tools that are very short lived in the system and makes dangerous overdosing essentially impossible, as the active compounds (peptides and auto inactivating or naturally degrading proteins) never accumulate, rapidly clear from the circulations in hours, restoring the full functioning of the immune system and all of its components instantaneously.

The treatment consists of any one of the active components, any combination of two of the active components, any combination of 3 of active components, any combination of 4 active components, all 5 components of the active components that are intended to remedy the abnormality of the targets involved in the cytokine storm.

Particularly, it is disclosed a treatment or therapeutic composition for use in the treatment of COVID-19 and other cytokine storm associated disorders including either:
a) complement factor 3-targeting inhibitor of complement activation cascade
b) carboxypeptidase B enzyme
c) complement factor 5a receptor-targeting inhibitor of complement activation cascade
d) endothelin A receptor-targeting inhibitor of extravasation
e) bone morphogenetic protein

alone, or combinations of 2, 3, 4 or 5 of said active agent a) to e), such as
a) with either b), c), d) or e),
b) with either c), d) or e),
c) with either d) or e), or
d) with e),

or combinations of
a) with either b) and c), or with b) and d), or with b) and e),
a) with either c) and d), or with c) and e),
a) with d) and e),
b) with either c) and d), or with c) and e), or with d) and e),
c) with either d) and e),

or combinations of
a) with either b), c) and d), or with c), d) and e),
b) with either a), c) and e), or with a), d) and e),
c) with either a), b) and d), or with b), d) and e),

or the combination of
a) with b), c), d) and e).

The therapeutic composition of the present invention provides means for effectively treating COVID-19 and other cytokine storm associated disorders. In some embodiments of the invention the therapeutic composition used comprises at least two, three, four or five distinct active agents being selected from at least two, three, four or five of the active agent groups a) to e).

The treatment includes in some embodiments a formulation for delivery as a pH stabilized infusion that counteracts potential tissue acidification. In some embodiments the inventive composition is formulated with antiviral, anti-microbial agents. The inventive composition may be delivered as preventative intervention or as treatment for destructive cytokine storm.

The delivery of the active compounds of the treatment for controlling the cytokine storm may coincide with the need to use optional factors, depending on the root cause of the cytokine storm. Since the treatment coincides with the treatment of the root cause, we include additional supporting material to be added in the final embodiment of the invention.
In case of virus infection, antiviral treatment may be included. (In preferred implementation it is hydroxy chloroquine supplemented with Azithromycin and Zinc/Selenium).
In case of septic cases, properly selected antibiotics may be added.
In case of hemorrhagic shock, volume extenders, erythrocytes or other blood substitute may be added.
In case of microthrombus formation, anti-coagulants may be included
In case of adverse tissue acidification due to insufficient oxygen supply is a severe complicating factor and, therefore, high buffer capacity of bicarbonate may be added to the treatment to reduce the destructive effects of the acidification such as uncontrolled disseminated complement activation.

### EXAMPLES

After selecting the targets, the inventor has designed and developed a series of highly specific compounds that meet the needs envisioned for the treatment of the overactive cytokine storm and break its positive feedback loops as well as moderate the tissue damage that it causes in the affected patients. As an additional requirement, the inventor selected a series of solution relaying on short lived compound that do not accumulate, break down naturally without leaving potentially toxic molecules behind.

### a) Complement Factor 3-Targeting Inhibitor of Complement Activation Cascade for Target 1

The inventor has designed a series of 19 peptides using a proprietary algorithm and the material composition is presented in table 1 below.

The peptides were synthesized by Fmoc solid phase method, purified on C18 reversa phase chromatography, tested by adding each peptide to and screened for complement inhibitory activity, in a simple antibody dependent hemolysis system in which 1 μg/mL of peptides were added to guinea pig complement, and were titrated against turkey erythrocytes mixed with hemolysin, a complement fixing anti-erythrocyte antibody solution. In the presence the peptides, the hemolythic activity of the complement was reduced, and the corresponding dilutions was determined. The inhibition was estimated by a formula:

$$Inhibition ( \% ) = 100 - ( dilution of sample titer * 100 ) / dilution of control$$

TABLE 1
List of peptides and their complement
inhibiting activity in a standard
complement mediated hemolysis assay.

| | | | Hemolysis |
|---|---|---|---|
| | One letter | SEQ | inhibition |
| Name | code sequence | ID No. | (% of maximum) |
| AP1314 | APNHLLEVRV | 1 | >80 |
| LTA1309 | LTAYVVKVFSLA | 2 | >80 |
| DDL1619 | DDLKQLANGVDRYI | 3 | >80 |
| SDK1606 | SDKKGICVADPFEVT | 4 | >80 |
| SDA984 | SDAGLTFTSS | 5 | >80 |
| SDK1709 | SDKKGICVADPCFEVT | 6 | >80 |
| VQA1857 | VQAERSGIPIVTSPYQI | 7 | >80 |
| QAL1363 | QALPYSTVGNSNL | 8 | >40-60 |
| NIF1464 | NIFLKDSITTWE | 9 | >40-60 |
| LHL1431 | LHLSVRTLELRP | 10 | >40-60 |
| QRS1008 | QRSYTVAIA | 11 | >40-60 |
| KRP1057 | KRPQDAKNT | 12 | >20-40 |
| ERL1041 | ERLGREGVQ | 13 | >20-40 |
| LNC1538 | LNCQRYYGGGGYST | 14 | >20-40 |
| LSR1099 | LSRKVLLDGV | 15 | >20-40 |
| ESA1331 | ESASLRSEETKV | 16 | >20-40 |
| GQW1513 | GQWKIRAYYENS | 17 | ≤20 |
| IAV1501 | IAVHYLDETEQW | 18 | ≤20 |
| LRL1384 | LRLPYVVREQL | 19 | ≤20 |
| TAY1409 | TAYVVKVFSLAVN | 20 | ≤20 |

### b) Carboxypeptidase B Enzyme For the Target 2

By rapid activation of carboxypeptidase B with trypsin, the inventor has found that the carboxypeptidase B enzyme activity is very low in plasma. What is more, it is rapidly depleted as infection, hemorrhage, hypoxia develops, blood coagulation triggered (cf. FIG. 1), in each case when systemic complement activation occurs, resulting in allowing anaphylatoxins to act at their full potential to trigger the positive feedback that results in Cytokine storm.

Further experiments indicated that, in rats, the carboxypeptidase B activity is completely depleted during the early phase of hemorrhagic shock model in rats (in 100 minutes it is reduced nondetectable levels). Providing external carboxypeptidase B and restoring the depletion could significantly slow down the events leading to the onset of lethal cytokine storm.

To replenish the carboxypeptidase pool, the inventor designed a recombinant human zinc-metalloproteinase, using the gene for pre-procarboxypeptidase B, encoded by cpb1 gene. The deduced protein the inventor edited the deduced protein sequence, replaced the promoter with a proprietary one that is optimizes for fast and effective secretion of the preproprotein, and introduced a mutation that replaced the original T with a N amino acid at the activation cleavage site and reduced the intracellular toxicity of the preproprotein that enables the overexpression with rapid secretion and results in high yield production. The inventor left the internal inactivation site intact, therefore retained the expected natural inactivation times.

In another embodiment of the invention, human carboxypeptidase B2 (TAFI) is used for its highly similar enzyme activity. This enzyme has been reengineered in order to add a novel, improved secretory peptide, reduced the strength of its activation signal that is needed to convert the pre pro enzyme into an active enzyme as well as reduced the availability of the cryptic tryptic cleavage site in order to prolong its half life in the serum.

In the case of carboxypeptidase B1, the sequence was reverse translated into DNA and optimized for high yield overexpression in DG44 cells. A standard EF1 promoter was added for high efficiently and constitutive expression and built into a payload plasmid that is used for making a lentiviral expression vector to transduce a cell line.

The expression cassette designed for the recombinant human carboxypeptidase preproprotein expression is shown in FIG. 3. The expression cassette uses a plasmid backbone and resides the 3′-5′ LTR regions. It contains a packaging signal (PSY) followed by a strong constitutive promoter (EF1 alpha). A new and improved leader peptide with improved cleavage site that enhances its secretion, followed by the optimized recombinant Carboxypeptidase B1 DNA sequence with triple stop codons, and a unique enhancer sequence.

The detailed specification of the obtained recombinant human carboxypeptidase B1 and B2 carboxypeptidase enzymes are shown in tables 2a and 2b below.

TABLE 2a
Specification of recombinant
human carboxypeptidase B1 enzyme

| Item | Value | Comment |
|---|---|---|
| Name | CPB1 human | Protein |
| | carboxypeptidase B | symbol |
| Gene | 1360 | |
| ID in | | |
| NBCI | | |
| database | | |
| Locus | 3q24 | |
| Protein in | P15086 (CBPB1_HUMAN) | |
| UniProt | | |
| Databse | | |
| EC Number | 3.4.17.2 | |
| Complete | MLALLVLVTVALASAHHGGE | \ |
| wild | HFEGEKVFRVNVEDENHINI | |
| type | IRELASTTQIDFWKPDSVTQ | |
| protein | IKPHSTVDFRVKAEDTVTVE | |
| SEQ ID | NVLKQNELQYKVLISNLRNV | |
| No. 49 | VEAQFDSRVRATGHSYEKYN | |
| | KWETIEAWTQQVATENPALI | |
| | SRSVIGTTFEGRAIYLLKVG | |
| | KAGQNKPAIFMDCGFHAREW | |
| | ISPAFCQWFVREAVRTYGRE | |
| | IQVTELLDKLDFYVLPVLNI | |
| | DGYIYTWTKSRFWRKTRSTH | |
| | TGSSCIGTDPNRNFDAGWCE | |
| | IGASRNPCDETYCGPAAESE | |
| | KETKALADFIRNKLSSIKAY | |
| | LTIHSYSQMMIYPYSYAYKL | |
| | GENNAELNALAKATVKELAS | |
| | LHGTKYTYGPGATTIYPAAG | |
| | GSDDWAYDQGIRYSFTFELR | |
| | DTGRYGFLLPESQIRATCEE | |
| | TFLAIKYVASYVLEHLY | |
| Leader | MLALLVLVTVALASA | Wild type |
| SEQ ID | | leader |
| No. 50 | | peptide |
| Recombinant | MRAPAQIFGFLLLLFPGTCF | New Leader |
| Sequence | AHHGGEHFEGEKVFRVNVED | peptide |
| SEQ ID | ENHINIIRELASTTQIDFWK | Activation |
| No. 21 | PDSVTQIKPHSTVDFRVKAE | pep. |
| | DTVTVENVLKQNELQYKVLI | T→Q |
| | SNLRNVVEAQFDSRVRANGH | mutation |
| | SYEKYNKWETIEAWTQQVAT | Active |
| | ENPALISRSVIGTTFEGRAI | domain |
| | YLLKVGKAGQNKPAIFMDCG | |
| | FHAREWISPAFCQWFVREAV | |
| | RTYGREIQVTELLDKLDFYV | |
| | LPVLNIDGYIYTWTKSRFWR | |
| | KTRSTHTGSSCIGTDPNRNF | |
| | DAGWCEIGASRNPCDETYCG | |
| | PAAESEKETKALADFIRNKL | |
| | SSIKAYLTIHSYSQMMIYPY | |
| | SYAYKLGENNAELNALAKAT | |
| | VKELASLHGTKYTYGPGATT | |
| | IYPAAGGSDDWAYDQGIRYS | |
| | FTFELRDTGRYGFLLPESQI | |
| | RATCEETFLAIKYVASYVLE | |
| | HLY | |

TABLE 2b
Specification of recombinant
human carboxypeptidase B1 enzyme

| Item | Value | Comment |
|---|---|---|
| Name | CPB2 human | Protein |
| | carboxypeptidase | symbol |
| | B2, TAFI | |
| Gene ID in | Q96IY4 | |
| Uniprot | | |
| NBCI | | |
| database | | |
| ECC | EC:3.4.17.20 | |
| Protein in | | |
| UniProt | | |
| Database | | |
| EC Number | 3.4.17.2 | |
| Complete wild | MKLCSLAVLVPIVLFC | \ |
| type protein | EQHVFAFQSGQVLAAL | |
| SEQ ID No. 51 | PRTSRQVQVLQNLTTT | |
| | YEIVLWQPVTADLIVK | |
| | KKQVHFFVNASDVDNV | |
| | KAHLNVSGIPCSVLLA | |
| | DVEDLIQQQISNDTVS | |
| | PRASASYYEQYHSLNE | |
| | IYSWIEFITERHPDML | |
| | TKIHIGSSFEKYPLYV | |
| | LKVSGKEQTAKNAIWI | |
| | DCGIHAREWISPAFCL | |
| | WFIGHITQFYGIIGQY | |
| | TNLLRLVDFYVMPVVN | |
| | VDGYDYSWKKNRMWRK | |
| | NRSFYANNHCIGTDLN | |
| | RNFASKHWCEEGASSS | |
| | SCSETYCGLYPESEPE | |
| | VKAVASFLRRNINQIK | |
| | AYISMHSYSQHIVFPY | |
| | SYTRSKSKDHEELSLV | |
| | ASEAVRAIEKTSKNTR | |
| | YTHGHGSETLYLAPGG | |
| | GDDWIYDLGIKYSFTI | |
| | ELRDTGTYGFLLPERY | |
| | IKPTCREAFAAVSKIA | |
| | WHVIRNV | |
| Leader | See Uniprot | Wild type |
| | Q96IY4 | leader |
| | | peptide |
| Recombinant | MRAPAQIFGFLLLLFP | New leader |
| Carboxypeptidase | GTCFAFQSGQVLAALP | Activation |
| B2 Sequence | RTSRQVQVLQNLTTTY | pep. |
| SEQ ID No. 22 | EIVLWQPVTADLIVKK | P→G mutation |
| | KQVHFFVNASDVDNVK | Cryptic |
| | AHLNVSGIPCSVLLAD | tryptic |
| | VEDLIQQQISNDTVSG | site |
| | RASASYYEQYHSLNEI | S→A |
| | YSWIEFITERHPDMLT | Active site |
| | KIHIGSSFEKYPLYVL | |
| | KVSGKEQTAKNAIWID | |
| | CGIHAREWISPAFCLW | |
| | FIGHITQFYGIIGQYT | |
| | NLLRLVDFYVMPVVNV | |
| | DGYDYSWKKNRMWRKN | |
| | RSFYANNHCIGTDLNR | |
| | NFASKHWCEEGASSSS | |
| | CSETYCGLYPESEPEV | |
| | KAVASFLRRNINQIKA | |
| | YISMHSYSQHIVFPYS | |
| | YTRAKSKDHEELSLVA | |
| | SEAVRAIEKTSKNTRY | |
| | THGHGSETLYLAPGGG | |
| | DDWIYDLGIKYSFTIE | |
| | LRDTGTYGFLLPERYI | |
| | KPTCREAFAAVSKIAW | |
| | HVIRNV | |

### c) C5a Receptor Inhibitor peptides for Target 3

The antisense homology box peptide corresponding to region 10-27 in the C5a receptor PR10 (DYGHYDDKDTLDLNTPVD; SEQ ID No. 26) did not inhibit the receptor activity, although it was demonstrated that it bound to two of its corresponding antisense peptides derived from C5a anaphylatoxin: peptides corresponding to amino acids 37-43 and 61-74.

The antisense homology box peptide PR101 (HWPFGGAACSILPSLI, SEQ ID No. 27, Mw: 1782 corresponding to region 10-27 in the C5a receptor) did not affect the C5a-induced Ca⁺⁺ influx; however, it was found that this peptide strongly interacted with the albumin present in the culture medium causing its precipitation and aggregation.

A peptide fragment of the C5a receptor corresponding to the loop between the fifth and sixth hypothetical transmembrane regions (amino acids 226-245) that is antisense to C5a and is an intramolecular AHB in C5a receptor proved to be a weak antagonist of C5a when preincubated with C5a at high concentrations (>0.5 μM). It was shown by its ability to inhibit Ca++ influx induced by C5a anaphylatoxin in C5a receptor-expressing (clAMP stimulated) U937 cells.

The same peptide fragment was shown to have C5a agonist activity as well, when U937 cells bearing the C5a receptor were preincubated with this peptide at a much lower concentration (even as little as 40 pM), the AHB peptide behaved as an agonist: Ca++ influx was efficiently triggered, even in the presence of an otherwise ineffective amount of C5a.

The AHB peptides derived from C5a anaphylatoxin, PL3 (QKKIEEIAAKYKHS, SEQ ID No. 52, Mw: 1670, C5a amino acids 3-16), PL12 (KYKHSVVKKSDGA, SEQ ID No. 53, Mw: 1801, C5a amino acids 12-27), PL37 (RAARISLGPRSIKAFTE, SEQ ID No. 54, Mw: 1985, C5a amino acids 37-51) and PL61 (LRANISHKDMQLGR, SEQ ID No. 55, Mw: 1740, C5a amino acids 61-74), were not able to inhibit C5a receptor in C5a-induced Ca++ influx assay.

The present invention includes methods and compositions that are designed to disable the ability of the C5a receptor to transmit cellular signals that are produced by binding of C5a to the receptor. One embodiment of the present invention is an inhibitory oligomer of peptides. One embodiment of the present invention consists of four repeats, the second consist of 8 monomers, collectively forming dendomer structure.

One specific monomer is a peptide of 17 amino acid long sequence derived from the C5a receptor itself. Each of the four monomers individually has a complex helical structure that forms spontaneously after the peptide is synthesized. Thus, the linear sequence of amino acids of each of the monomers which is manufactured in a peptide synthesizer at low cost, forms by itself into a more complex 3-dimensional structure, and the four or eight monomers are assembled during manufacture as a tetramer or octamer, or synthesized using a core in the form of
for tetrameric dendromer X-K-K₂-(peptide)₄
or
for Octameric dendromer: or X-K-K₂-K₄-(peptide): (SEQ ID No. 69).

It is to be understood that although an oligomer with four repeats of the peptide is the most desired form of the present invention, oligomers with 2 through 16 repeats of the peptide are also affective in inhibiting C5a receptors. Another embodiment of the oligomer is the octameric repeat of the peptide.

Because of the particularly folded conformation of each monomer, and because of the additive effects of the four monomers, the tetramer fits perfectly with high affinity within the inner folded structure of the C5a receptor and blocks the ability of the receptor to induce damaging biological effects. The damaging caused by C5a effects that can be prevented by this compound include: the activation of granulocytes and release of tissue-injuring enzymes, constriction of blood vessels and capillaries which severely restrict the perfusion of tissues by blood, inflammation that changes vascular permeability, causes edema, attracts cells which in turn provoke severe tissue damage, and other effects that promote acute extension of myocardial infarcts.

The present invention is a C5a inhibitor that is differentiated from other products that inhibit C5a activity (such as antibodies or other proteins or peptides), that this construct will not only bind to C5a, reducing and neutralizing the available amount of C5a, but in the same time it binds to the C5a receptor, inhibiting its activity as a true C5a receptor inhibitor as well. This double-sided activity results in extremely powerful and efficient C5a inhibitor in that it substantially inactivates the C5a receptor's ability to function as a receptor. Other strategies currently being developed attempt to compete with C5a for binding to the receptor. A beneficial consequence of the present invention is the requirement of a very low effective concentration of the inhibitor to prolong the term of prevention of the damage caused by C5a by prolonged disabling of the receptor's ability to function, while the low concentration of the present invention minimizes the potential side effects.

A distinct advantage of the present invention is that, because it inactivates the receptor itself, its effectiveness is not influenced by the local concentration of C5a. The inhibitor peptide is effective even in the presence of massive amounts of C5a that are typically generated during complement activation in the course of a heart attack or other adverse event. Other strategies that simply compete with C5a for binding to the C5a receptor can be overcome simply by increasing the local concentration of C5a to overwhelm the inhibitor. This inhibitor does not affect C5a binding and does not compete with the receptor-ligand interaction, rather affects the conformation of the C5aR and blocks its ability to transfer stimulatory information into the cell.

TABLE 3
List of C5a receptor inhibitor peptides

| | One letter | | | |
|---|---|---|---|---|
| Peptide | code | SEQ | | |
| ID | sequence | ID No. | | |
| LRT2463 | LRTWSRRATR | 23 | Monomeric | PR226 |
| | STKTLKVV | | | |
| LRT10353 | (LRTWSRRAT | 24, 43, | Tetrameric | PR226TET |
| | RSTKTLKV | and 44 | dendromer | |
| | V)₄K₂KV | | | |
| LRT20707 | (LRTWSRRAT | 25, 43, | Octameric | PR226MAP |
| | RSTKTLKV | 44, and | dendromer | |
| | V)₈K₄K₂KV | 45 | | |
| DYG2094 | DYGHYDDKDT | 26 | Monomeric | PR10 |
| | LDLNTPVD | | | |
| HWP1667 | HWPFGGAACS | 27 | Monomeric | PR101 |
| | ILPSLI | | | |

In FIG. 2 the activity is shown for PR226MAP, which is the most potent C5aR inhibitor. Particularly, it is shown the IC50 achieved at pM levels. Serial dilution of PR226 MAP peptide inhibited the C5a triggered Ca influx into the cells at femtomolar concentration levels, while the IC50 was as low as 1 pM. Complete inhibition was reached at 200 pM in this experiment. The reason is that the peptide immediately binds to the erythrocyte surfaces and forms a stable membrane bound pool, that is completely inert to erythrocytes, but upon contact with C5aR bearing cells, the inhibitory effect is fully exhibited. In addition, in the membrane bound state the peptide remains active for at least 12 hours. Therefore, it is a very effective C5aR inhibitor.

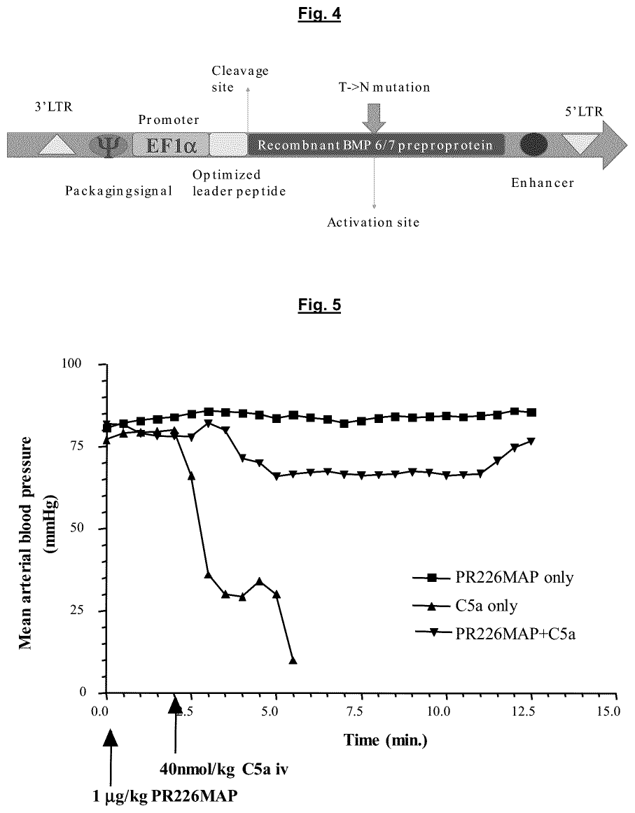

Further, it has been shown that the peptide can be delivered in vivo very safely, even at very high concentration. In FIG. 5 it is shown that an injection of 1 μg/kg PR226MAP peptide did not induce any cardiovascular change as represented by the same average blood pressure but abrogated the lethal effects of the subsequent challenge with the lethal dose of C5a.

FIG. 6 shows that the animal survived 7 injections but developed accute hemorrhage and succumbed by the third hour displaying all the symptoms of acute cytokine storm.

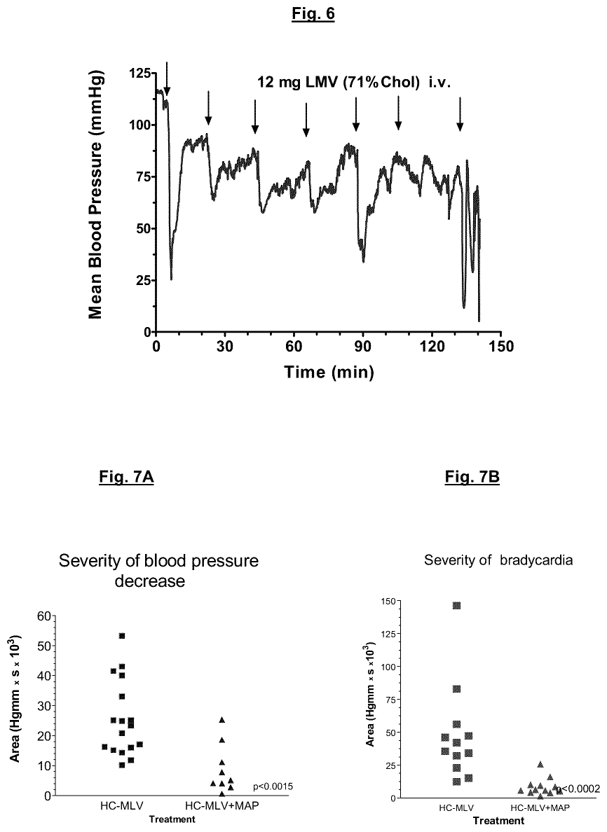

FIGS. 7A and 7B show that the C5a receptor inhibitor peptide protects rats from the cardiovascular effects of the cytokine storm. The C5aR inhibitor blocked the hemodynamic and lethal effects of complement activation as well as the subsequent cytokine storm that manifested in cardiac collapse: bradycardia and severe fall of blood pressure, often cited as the fatal signs of the Cytokine storm.

### d) The Endothelin Receptor Inhibitors for Target 4

The endothelin receptor is a complex of seven transmembrane helices and loops connecting them and belong to the family of the G-protein associated receptors the same family of receptors the C5a receptor is also a member. The inventor has run similar analysis and identified several antisense homology box peptides, using the same proprietary algorithm, and synthesis process (FMOC solid phase synthesis). The peptides and their variants have been produced and tested in standard functional endothelin assay in which external endothelin is added to prepared smooth muscles cells and the endothelin induced muscle contractility was measured. The peptides that inhibit the signal transduction by the receptor, are identified as Endothelin receptor inhibitors and are listed in Table 4 by their peptide ID, the amino acid sequence and their referenced name in the art.

TABLE 4
List of endothelin receptor A inhibiting peptides.

| Peptide | One letter | SEQ | |
|---|---|---|---|
| ID | code sequence | ID No. | Comment/name |
| CAL1595 | CALSVDRYRAVAS | 28 | ETR-P1 |
| | W | | |
| CAL1698 | CALSVDRYRAVAS | 29 | ETR-P1/cyc |
| | WC | | (cyclic) |
| QGI10797 | (QGIGIPLITAE | 30, 46, | ERT-P3MAP |
| | I)₈K₄K₂KV | 47, and | |
| | | 48 | |
| LNL2390 | LNLCALSVDRYRA | 31 | ETR-P1/fl |
| | VASWSRVI | | (extended) |
| IVR2333 | IVRSWSAVARYDS | 32 | ETR-P1/fl.re |
| | VLACLNLV | | (reversed |
| | | | sequence) |
| CAL2614 | CALSVDRYRAVAS | 33 | ETR-P1/fl.sp |
| | WGIPLITAIEI | | |
| VLN2390 | VLNLCALSVDRYG | 34 | ETR-P1/fl.m1 |
| | AVASWSRVI | | |
| VLN2433 | VLNLCAGSVDRYR | 35 | ETR-P1/fl.m2 |
| | AVASWSRVI | | |
| VLN2443 | VLNLGALSVDRYR | 36 | ETR-P1/fl.m3 |
| | AVASWSRVI | | |
| TDP610 | (tryptamine-) | 37 | BQ123 |
| | DPVL | | |

One of the peptides was subsequently tested in dogs and confirmed its biological effect in vivo. Injecting dogs iv with 50 nm of ET-1 peptide resulted in a rapid increase in the concentration of the peptide: the normal baseline values of 2-3 fM increased to as high as 10-15 fM within 1 min and remained at high level for at least 10 min, followed by slow but steady decrease, but even after 30 min, the circulating endothelin level exceeded 6-7 fM. The elevated ET-1 level was marked with profound changes in circulation (details to be published elsewhere). However, pretreatment of dogs with 100 ug/kg ETR-P1/fl peptide removed the cardiovascular effects of injected ET-1 peptide from the circulation within 10 min and the measured endothelin level become normal. In spite of strong “buffering” effect of ETR-P1/fl peptide on externally administered ET-1 our data indicate that the peptide had a little or no influence (except a transient, 30-50% decrease) on the normal baseline level of ET peptide in the plasma. The explanation of this finding is that this peptide changed the conformation of the endothelin receptor, and that became capable of binding 4 times more ET-1 peptide, while frozen in an inactive conformation as far as receptor activation is concerned, indicating its high specificity, potency and stability that it can be applied in vivo. It is known in the previous art and has been patented and the patent expired.

The inventor also found that endothelin A receptor-targeting inhibitor of extravasation peptides do actively prevent leukocyte extravasation from circulation when stimulated by ET-1 in experimental system induced firm leukocyte adherence throughout the 90-minute observation period in animal models. The ETB-receptor antagonist IRL 1038 did not influence the number of sticking leukocytes in the submucosal postcapillary venules but transiently reduced the number of stickers in the collecting venules 30 minutes after ET-1 administration.

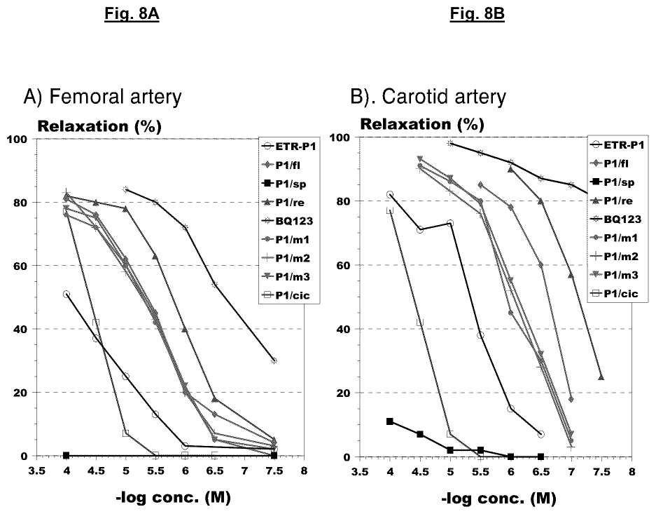

FIGS. 8A and 8B show the effective inhibition of smooth muscle contraction by the inventive peptides capable of blocking ET-A receptors.

### e) The Recombinant Bone Morphogenetic Protein 6 for Target 5

By screening over 30,000 clones including the unmodified BMP6 expression system a 2-5 mg/L volumetric yield could be achieved. Therefore, the inventor designed a recombinant bone morphogenetic protein combining the fibroblast blocking and wound healing promoting effect of BMP 6 by molecular engineering. The protein was modified with novel leader peptide that was developed by the inventor. This new leader peptide improves secretion 4 times. Further, a modified activation site was engineered that reduces the activation and prevents the enzyme to be activated inside the cells and reduces its toxicity when overexpressed. With these modifications, extraordinary expression rates were achieved. Particularly, by screening of less than 180 clones, including the new recombinant bone morphogenetic protein, over 1 g/L volumetric productivity was achieved in a bioreactor. This allows the production of over 500-1000 purified human doses in a single mL of supernatant.

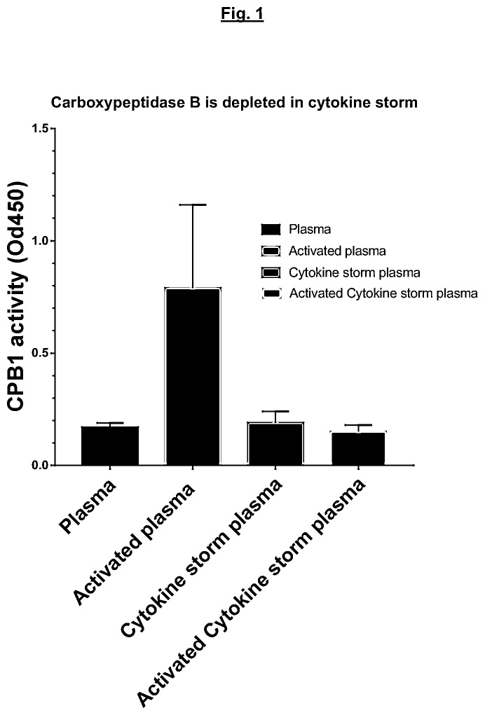

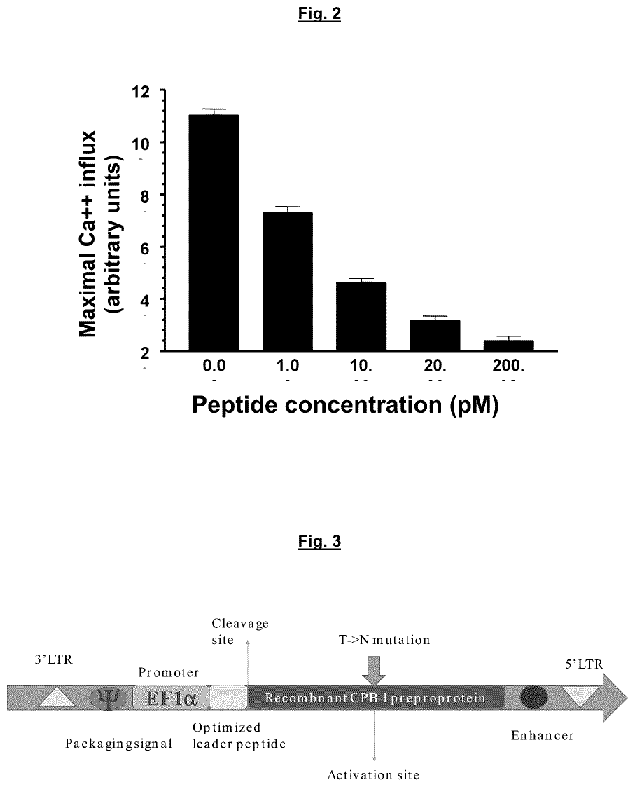

The expression cassette designed for the recombinant bone morphogenetic protein is shown in FIG. 4. The expression cassette uses a plasmid backbone and resides the 3′-5′ LTR regions. It contains a packaging signal (PSY) followed by a strong constitutive promoter (EF1 alpha). A new and improved leader peptide with improved cleavage site that enhances its secretion, followed by the optimized recombinant bone morphogenetic protein 6 sequence with triple stop codons, and a unique enhancer sequence.

The material composition of the recombinant human Bone morphogenetic protein 6 (BMP6) has been improved by adding an optimized promoter and editing the sequence to reduce self-activation and the subsequent spontaneous inactivation of the protein by introducing a novel mutation at the activation site RRQ [symbol] ->RRQ [symbol] (SEQ ID Nos: 61 and 62, respectively).

TABLE 5
Specifications of recombinant human
bone morphogenetic protein 6 (BMP6)

| Protein item | Value | Comment |
|---|---|---|
| Wild type BMP-6 | MAPFEPLASGILLLLWLICGPPPLR | Gene |
| protein | PPLPAAAAAAAGGQLLGDGGSPGRT | BMP6 |
| sequence | EQPPPSPQSSSGFLYRRLKTQEKRE | |
| (BMP6_HUMAN) | MQKEILSVLGLPHRPRPLHGLQQPQ | |
| SEQ ID No. 56 | PPALRQQEEQQQQQQLPRGEPPPGR | |
| | LKSAPLFMLDLYNALSADNDEDGAS | |
| | EGERQQSWPHEAASSSQRRQPPPGA | |
| | AHPLNRKSLLAPGSGSGGASPLTSA | |
| | QDSAFLNDADMVMSFVNLVEYDKEF | |
| | SPRQRHHKEFKFNLSQIPEGEVVTA | |
| | AEFRIYKDCVMGSFKNQTFLISIYQ | |
| | VLQEHQHRDSDLFLLDTRVVWASEE | |
| | GWLEFDITATSNLWVVTPQHNMGLQ | |
| | LSVVTRDGVHVHPRAAGLVGRDGPY | |
| | DKQPFMVAFFKVSEVHVRTTRSASS | |
| | RRRQQSRNRSTQSQDVARVSSASDY | |
| | NSSELKTACRKHELYVSFQDLGWQD | |
| | WIIAPKGYAANYCDGECSFPLNAHM | |
| | NATNHAIVQTLVHLMNPEYVPKPCC | |
| | APTKLNAISVLYFDDNSNVILKKYR | |
| | NMVVRACGCH | |
| Old leader | MAPFEPLASGILLLLWLI | |
| peptide | | |
| SEQ ID No. 57 | | |
| New leader | MPGLGRRAQWLCWWWGLLCSC | Optimized |
| peptide | | |
| SEQ ID No. 58 | | |
| Propeptide | CGPPPLRPPLPAAAAAAAGGQLLGD | |
| SEQ ID No. 59 | GGSPGRTEQPPPSPQSSSGFLYRRL | |
| | KTQEKREMQKEILSVLGLPHRPRPL | |
| | HGLQQPQPPALRQQEEQQQQQQLPR | |
| | GEPPPGRLKSAPLFMLDLYNALSAD | |
| | NDEDGASEGERQQSWPHEAASSSQR | |
| | RQPPPGAAHPLNRKSLLAPGSGSGG | |
| | ASPLTSAQDSAFLNDADMVMSFVNL | |
| | VEYDKEFSPRQRHHKEFKFNLSQIP | |
| | EGEVVTAAEFRIYKDCVMGSFKNQT | |
| | FLISIYQVLQEHQHRDSDLFLLDTR | |
| | VVWASEEGWLEFDITATSNLWVVTP | |
| | QHNMGLQLSVVTRDGVHVHPRAAGL | |
| | VGRDGPYDKQPFMVAFFKVSEVHVR | |
| | TTRSASSRRR | |
| Active domain | QQSRNRSTQSQDVARVSSASDYNSS | |
| SEQ ID No. 60 | ELKTACRKHELYVSFQDLGWQDWII | |
| | APKGYAANYCDGECSFPLNAHMNAT | |
| | NHAIVQTLVHLMNPEYVPKPCCAPT | |
| | KLNAISVLYFDDNSNVILKKYRNMV | |
| | VRACGCH | |
| Cleavage site | RRQQ→RRQA | Weakened |
| Mutation | | cleavage |
| SEQ ID Nos. 61 | | site |
| and 62 | | |
| Complete | MPGLGRRAQWLCWWWGLLCSCCGPP | |
| Recombinant | PLRPPLPAAAAAAAGGQLLGDGGSP | |
| Human BMP-6 | GRTEQPPPSPQSSSGFLYRRLKTQE | |
| SEQ ID No. 38 | KREMQKEILSVLGLPHRPRPLHGLQ | |
| | QPQPPALRQQEEQQQQQQLPRGEPP | |
| | PGRLKSAPLFMLDLYNALSADNDED | |
| | GASEGERQQSWPHEAASSSQRRQPP | |
| | PGAAHPLNRKSLLAPGSGSGGASPL | |
| | TSAQDSAFLNDADMVMSFVNLVEYD | |
| | KEFSPRQRHHKEFKFNLSQIPEGEV | |
| | VTAAEFRIYKDCVMGSFKNQTFLIS | |
| | IYQVLQEHQHRDSDLFLLDTRVVWA | |
| | SEEGWLEFDITATSNLWVVTPQHNM | |
| | GLQLSVVTRDGVHVHPRAAGLVGRD | |
| | GPYDKQPFMVAFFKVSEVHVRTTRS | |
| | ASSRRRQASRNRSTQSQDVARVSSA | |
| | SDYNSSELKTACRKHELYVSFQDLG | |
| | WQDWIIAPKGYAANYCDGECSFPLN | |
| | AHMNATNHAIVQTLVHLMNPEYVPK | |
| | PCCAPTKLNAISVLYFDDNSNVILK | |
| | KYRNMVVR | |

Further, secretory and membrane attached forms of the recombinant human Bone Morphogenetic protein have been constructed.

TABLE 6
Specifications of recombinant human
Bone morphogenetic protein 6 (BMP6)

| Item | Value |
|---|---|
| Wilde type human BMP 6 | MPGLGRRAQWLCWWWGLLCSCC |
| >sp\|P22004\|BMP6_HUMAN | GPPPLRPPLPAAAAAAAGGQLL |
| Bone morphogenetic protein | GDGGSPGRTEQPPPSPQSSSGF |
| 6 OS = Homo sapiens | LYRRLKTQEKREMQKEILSVLG |
| OX = 9606 GN = BMP6 PE = 1 | LPHRPRPLHGLQQPQPPALRQQ |
| SV = 1 | EEQQQQQQLPRGEPPPGRLKSA |
| SEQ ID No. 63 | PLFMLDLYNALSADNDEDGASE |
| | GERQQSWPHEAASSSQRRQPPP |
| | GAAHPLNRKSLLAPGSGSGGAS |
| | PLTSAQDSAFLNDADMVMSFVN |
| | LVEYDKEFSPRQRHHKEFKFNL |
| | SQIPEGEVVTAAEFRIYKDCVM |
| | GSFKNQTFLISIYQVLQEHQHR |
| | DSDLFLLDTRVVWASEEGWLEF |
| | DITATSNLWVVTPQHNMGLQLS |
| | VVTRDGVHVHPRAAGLVGRDGP |
| | YDKQPFMVAFFKVSEVHVRTTR |
| | SASSRRRQQSRNRSTQSQDVAR |
| | VSSASDYNSSELKTACRKHELY |
| | VSFQDLGWQDWIIAPKGYAANY |
| | CDGECSFPLNAHMNATNHAIVQ |
| | TLVHLMNPEYVPKPCCAPTKLN |
| | AISVLYFDDNSNVILKKYRNMV |
| | VRACGCH |
| Wilde type human BMP7 | MHVRSLRAAAPHSFVALWAPLF |
| >sp\|P18075\|BMP7_HUMAN | LLRSALADFSLDNEVHSSFIHR |
| Bone morphogenetic protein | RLRSQERREMQREILSILGLPH |
| 7 OS = Homo sapiens | RPRPHLQGKHNSAPMFMLDLYN |
| OX = 9606 GN = BMP7 PE = 1 | AMAVEEGGGPGGQGFSYPYKAV |
| SV = 1 | FSTQGPPLASLQDSHFLTDADM |
| SEQ ID No. 64 | VMSFVNLVEHDKEFFHPRYHHR |
| | EFRFDLSKIPEGEAVTAAEFRI |
| | YKDYIRERFDNETFRISVYQVL |
| | QEHLGRESDLFLLDSRTLWASE |
| | EGWLVFDITATSNHWVVNPRHN |
| | LGLQLSVETLDGQSINPKLAGL |
| | IGRHGPQNKQPFMVAFFKATEV |
| | HFRSIRSTGSKQRSQNRSKTPK |
| | NQEALRMANVAENSSSDQRQAC |
| | KKHELYVSFRDLGWQDWIIAPE |
| | GYAAYYCEGECAFPLNSYMNAT |
| | NHAIVQTLVHFINPETVPKPCC |
| | APTQLNAISVLYFDDSSNVILK |
| | KYRNMVVRACGCH |
| Wilde type leader peptide | MPGLGRRAQWLCWWWGLLCS |
| SEQ ID No. 65 | (removed) |
| Optimized leader peptide | MRAPAQIFGFLLLLFPGTCFA |
| SEQ ID No. 66 | (added) |
| Recombinant BMP7 active | CCGPPPLRPPLPAAAAAAAGGQ |
| domain I with inserts from | LLGDGGSPGRTEQPPPSPQSSS |
| BMP6 | GFLYRRLKTQEKREMQKEILSV |
| SEQ ID No. 41 | LGLPHRPRPLHGLQQPQPPALR |
| | QQEEQQQQQQLPRGEPPPGRLK |
| | SAPLFMLDLYNALSADNDEDGA |
| | SEGERQQSWPHEAASSSQRRQP |
| | PPGAAHPLNRKSLLAPGSGSGG |
| | ASPLTSAQDSAFLNDADMVMSF |
| | VNLVEYDKEFSPRQRHHKEFKF |
| | NLSQIPEGEVVTAAEFRIYKDC |
| | VMGSFKNQTFLISIYQVLQEHQ |
| | HRDSDLFLLDTRVVWASEEGWL |
| | EFDITATSNLWVVTPQHNMGLQ |
| | LSVVTRDGVHVHPRAAGLVGRD |
| | GPYDKQPFMVAFFKVSEVHVRT |
| | TRSASSRRR |
| Active domain II | QQSRNRSTQSQDVARVSSASDY |
| SEQ ID No. 42 | NSSELKTACRKHELYVSFQDLG |
| | WQDWIIAPKGYAANYCDGECSF |
| | PLNAHMNATNHAIVQTLVHLMN |
| | PEYVPKPCCAPTKLNAISVLYF |
| | DDNSNVILKKYRNMVVRACGCH |
| Human IgG Hinge region | RWPESPKAQASSVPTAQPQAEG |
| SEQ ID No. 67 | SLAKATAPATTRNTGRGGEEKK |
| | KEKEKEEQEERETKTPECP |
| Human CD59 PI anchor | QCYNCPNPTADCKT |
| SEQ ID No. 68 | AVNCSSDFDA CLITKAELGY |
| | HYVAQAGRRQ SSHFSLLKCW |
| | DYRCEPSHWP |
| | HCPYFNWGYKCITSVGSLSI |
| | AISTTSQPA |
| Complete recombinant | MRAPAQIFGFLLLLFPGTCFAC |
| BMP sequence for | CGPPPLRPPLPAAAAAAAGGQL |
| secretory production | LGDGGSPGRTEQPPPSPQSSSG |
| SEQ ID No. 39 | FLYRRLKTQEKREMQKEILSVL |
| | GLPHRPRPLHGLQQPQPPALRQ |
| | QEEQQQQQQLPRGEPPPGRLKS |
| | APLFMLDLYNALSADNDEDGAS |
| | EGERQQSWPHEAASSSQRRQPP |
| | PGAAHPLNRKSLLAPGSGSGGA |
| | SPLTSAQDSAFLNDADMVMSFV |
| | NLVEYDKEFSPRQRHHKEFKFN |
| | LSQIPEGEVVTAAEFRIYKDCV |
| | MGSFKNQTFLISIYQVLQEHQH |
| | RDSDLFLLDTRVVWASEEGWLE |
| | FDITATSNLWVVTPQHNMGLQL |
| | SVVTRDGVHVHPRAAGLVGRDG |
| | PYDKQPFMVAFFKVSEVHVRTT |
| | RSASSRRRQQSRNRSTQSQDVA |
| | RVSSASDYNSSELKTACRKHEL |
| | YVSFQDLGWQDWIIAPKGYAAN |
| | YCDGECSFPLNAHMNATNHAIV |
| | QTLVHLMNPEYVPKPCCAPTKL |
| | NAISVLYFDDNSNVILKKYRNM |
| | VVRACGCH |
| Complete recombinant | MRAPAQIFGFLLLLFPGTCFAQ |
| BMP sequence for cell | QSRNRSTQSQDVARVSSASDYN |
| surface and exsosome | SSELKTACRKHELYVSFQDLGW |
| expression | QDWIIAPKGYAANYCDGECSFP |
| SEQ ID No. 40 | LNAHMNATNHAIVQTLVHLMNP |
| | EYVPKPCCAPTKLNAISVLYFD |
| | DNSNVILKKYRNMVVRACGCHR |
| | WPESPKAQASSVPTAQPQAEGS |
| | LAKATAPATTRNTGRGGEEKKK |
| | EKEKEEQEERETKTPECPQCYN |
| | CPNPTADCKTAVNCSSDFDACL |
| | ITKAELGYHYVAQAGRRQSSHF |
| | SLLKCWDYRCEPSHWPHCPYFN |
| | WGYKCITSVGSLSIAISTTSQP |
| | A |

### ABBREVIATIONS AND DEFINITIONS

| Term | Definition/comment |
|---|---|
| nCOV- | New coronavirus or SARS nCOV-2 coronavirus |
| | causing COVID-19 disease |
| CoVid-19 | The disease caused by the SARS nCOV-2 virus |
| ARDS | Acute respiratory disease |
| MERS | Middle Eastern Respiratory Syndrome Virus |
| SARS | Severe Acute Respiratory syndrome Virus |
| Spike protein | Surface glycoprotein of Severe acute |
| | respiratory syndrome [nCOV-2 |
| C5aR | C5a Receptor |
| CD88 | C5a Receptor |
| C5a | Complement factor 5 derived anaphylatoxin |
| C4a | Complement factor 4 derived anaphylatoxin |
| C3a | Complement factor 3 derived anaphylatoxin |
| IV IgG | Intravenous Immunoglobulin G |
| PIF | Phagocytosis Inhibitor Factor |

The inventive peptides disclosed herein are defined by their amino acid sequence as presented respectively. The present invention also includes the following modifications thereof.

Qa—place amino acids here—Qc″, wherein Q″ is selected from the group consisting of hydrogen, alkyl, aryl, arylalkyl, alkyloyl, aryloyl, arylalkyloyl, alkyloxoyl, aryloxoyl and arylalkyloxoyl; and QC is selected from the group consisting of hydroxy, alky loxy, aryloxy, arylalkyloxy, alkylamino, dialkylamino, ary lamino, diarylamino, (aryl) (alkyl) amino, arylalkylamino, diarylalkylamino, (alkyl) (arylalkyl) amino, (aryl) (arylalky I) amino, and amino. Preferably Qa″ is hydrogen and QC is hydroxy, i.e., H—place amino acids here—OH. Preferably Q″ is H3C—C(O)— and QC is amino, i.e., H3C—C(O)——place amino acids here—NHZ. In another embodiment of the invention, the anti-wrinkle agent is a peptide having the amino acid sequence Qa—amino acids—Q″, wherein Q″ is selected from the group. In addition a peptide can be synthesis in dendromer form, where the peptide is described by the (Qa—place amino acids here—Q)₈K₄K₂K-x formula of dendromeric branching structure. With Qa consisting of hydrogen, alkyl, aryl, arylalkyl, alkyloyl, ary˜loyl, arylalkyloyl, alkyloxoyl, aryloxoyl and arylalkyloxoyl; and QC is selected from the group consisting of hydroxy, alkyloxy, aryloxy, arylalkyloxy, alkylamino, dialkylamino, arylamino, diarylarnino, (aryl) (alkyl) amino, arylalkylamino, diarylalkylamino, (alkyl) (arylalkyl) amino, (aryl) (arylalky I) amino, and amino. Preferably Qa, is hydrogen and QC is hydroxy.

To obtain the proper list of derivatives, replace the place holder amino acid sequence with each the listed amino acid sequence of the inhibitor peptides claimed in this invention.

As used herein, “alkyl” and “R” mean a hydrocarbon chain which may be straight, or branched; substituted (mono-or poly-) or unsubstituted, preferably unsubstituted; saturated or monounsaturated (i.e., one double or triple bond in the chain), or polyunsaturated (i.e., two or more double bonds in the chain, two or more triple bonds in the chain, or one or more double and one or more triple bonds in the chain). As used herein, “aryl” and “Ar” mean an aromatic; substituted (mono-or poly-) or unsubstituted, preferably unsubstituted. Preferred aryls are phenyl, pyridyl, pynnidyl and napthyl; more preferred is phenyl. As used herein, “arylalkyl” means Ar—R—. As used herein, “alkyloyl” means R—C(O)— As used herein, “aryloyY” means Ar—C(O—. As used herein, “arylalkyloyl” means Ar—R—C(O)—. As used herein, “alkyloxoyl” means R—O—C(O)—. As used herein, “aryloxoyl” means Ar—O—C(O)—. As used herein, “arylalkyloxoyl” means Ar—R—O—C(O)—. As used herein, “alkyloxy” means R—O—. As used herein, “aryloxy” means Ar—O—. As used herein, “arylalkyloxy” means Ar—R—O—. As used herein, “alkylamino” means R—N(H)—. As used herein, “dialkylamino” means R2N—. As used herein, “arylamino” means Ar—N(H)—. As used herein, “diarylamino” means (Ar)2N—. As used herein, “(aryl) (alkyl) amino” means Ar—N(R)—. As used herein, “arylalkylamino” means Ar—R—N (H)—. As used herein, “diarylalkylamino” means (Ar—R)2N—. As used herein, “(alkyl) (arylalkyl) amino” means Ar—R—N(R)—. As used herein, “(aryl) (arylalkyl) amino” means Ar—R—N(R)—.

1. A therapeutic composition comprising at least one active agent selected from active agent groups a) to e):
a) complement factor 3-targeting inhibitors;
b) carboxypeptidase B enzymes;
c) complement factor 5a receptor-targeting inhibitors;
d) endothelin A receptor-targeting inhibitors;
e) bone morphogenic proteins.

2. The therapeutic composition according to claim 1, wherein the therapeutic composition comprises at least two, three, four or five distinct active agents being selected from at least two, three, four or five of the active agent groups a) to e).

3. The therapeutic composition according to claim 1, wherein the complement factor 3-targeting inhibitor of active agent group a) is selected from a group of peptides having amino acid sequences as specified in SEQ ID Nos. 1-20.

4. The therapeutic composition according to claim 1, wherein the carboxypeptidase B enzyme of active agent group b) is selected from carboxypeptidase B (EC 3.4.16-3.4.18), or a recombinant carboxypeptidase B enzyme having an amino acid sequence of SEQ ID No. 21 or SEQ ID No. 22.

5. The therapeutic composition according to claim 1, wherein the complement factor 5a receptor-targeting inhibitor of active agent group c) is selected from a group of peptides having amino acid sequences as specified in SEQ ID Nos. 23-27.

6. The therapeutic composition according to claim 1, wherein the endothelin A receptor-targeting inhibitor of active agent group d) is selected from a group of peptides having amino acid sequences as specified in SEQ ID Nos. 28-37.

7. The therapeutic composition according to claim 1, wherein the bone morphogenic protein of active agent group e) is selected from bone morphogenic protein BMP-6, or a recombinant bone morphogenic protein BMP having an amino acid sequence as specified in one of the SEQ ID Nos. 38 to 40 or comprising an active domain having an amino acid sequence as specified in one of the SEQ ID Nos. 41 or 42.

8. A recombinant bone morphogenic protein BMP having an amino acid sequence as specified in one of the SEQ ID Nos. 38 to 40 or comprising an active domain having an amino acid sequence as specified in one of the SEQ ID Nos. 41 or 42.

9. A nucleic acid encoding the recombinant bone morphogenic protein BMP of claim 8.

10. A plasmid or lentiviral vector comprising the nucleic acid of claim 9.

11. A cell line comprising the plasmid of claim 10.

12. A method of producing recombinant bone morphogenic protein BMP, comprising cultivating the cell line according to claim 11.

13. A method of treating a cytokine storm associated disorder in a subject in need thereof, comprising administering to the subject an effective amount of at least one active agent selected from active agent groups a) to e):
a) complement factor 3 targeting inhibitor of complement activation cascade
b) carboxypeptidase B enzyme
c) complement factor 5a receptor targeting inhibitor of complement activation cascade
d) endothelin A receptor targeting inhibitor of extravasation
e) bone morphogenic protein.

14. The method according to claim 13, wherein the complement factor 3-targeting inhibitor of active agent group a) is selected from a group of peptides having amino acid sequences as specified in SEQ ID Nos. 1-20.

15. The method according to claim 13, wherein the carboxypeptidase B enzyme of active agent group b) is selected from carboxypeptidase B (EC 3.4.16-3.4.18), or a recombinant carboxypeptidase B enzyme having an amino acid sequence of SEQ ID No. 21 or SEQ ID No. 22.

16. The method according to claim 13, wherein the complement factor 5a receptor-targeting inhibitor of active agent group c) is selected from a group of peptides having amino acid sequences as specified in SEQ ID Nos. 23-27.

17. The method according to claim 13, wherein the endothelin A receptor-targeting inhibitor of active agent group d) is selected from a group of peptides having amino acid sequences as specified in SEQ ID Nos. 28-37.

18. The method according to claim 13, wherein the bone morphogenic protein of active agent group e) is selected from bone morphogenic protein BMP-6, or a recombinant bone morphogenic protein BMP having an amino acid sequence as specified in one of the SEQ ID Nos. 38 to 40 or comprising an active domain having an amino acid sequence as specified in one of the SEQ ID Nos. 41 or 42.
